(19) United States
(12) Patent Application Publication
(10) Pub. No.: US 2010/0286188 A1
(43) Pub. Date: Nov. 11, 2010

(54) MEANS FOR IMPROVING COGNITIVE FUNCTIONS AND MEMORY BASED ON HYDROGENATED PYRIDO(4,3-B)INDOLES (VARIANTS), PHARMACOLOGICAL MEANS BASED THEREON AND METHOD FOR THE USE THEREOF

(76) Inventors: Sergey O. Bachurin, Chernogolovka (RU); Svetlana I. Gavrilova, Moscow (RU); Vladimir V. Grigoriev, Chernogolovka (RU); Bogdan K. Beznosko, Chernogolovka (RU); Nikolay S. Zefirov, Moscow (RU)

Correspondence Address: MORRISON & FOERSTER LLP, 755 PAGE MILL RD, PALO ALTO, CA, 94304-1018, US

(21) Appl. No.: 12/517,191

(22) PCT Filed: Nov. 30, 2007
(86) PCT No.: PCT/US07/24623

§ 371 Date: Jul. 22, 2010

(30) Foreign Application Priority Data
Dec. 1, 2006 (RU) 2006142521

(51) Int. Cl. A61K 31/437; C07D 471/04; A61P 25/00; A61P 25/28

(52) U.S. Cl. 514/292; 546/85

(57) ABSTRACT

A means for improving cognitive functions and memory based on hydrogenated pyrido (4,3-b) indoles (variants), a pharmacological means based thereon and a method for the use thereof relate to the use of chemical compounds in the field of medicine and may be used to extend the arsenal of means which can be utilized for substantial enhancement of memory and activation of the learning process, for the treatment of impaired cognitive functions and memory in the elderly and in mild cognitive impairment, in brain trauma, in chronic cerebrovascular insufficiency, in hypoxic encephalopathies, resulting from chronic alcoholism and in delayed development in children. Said task is resolved by the use of hydrogenated pyrido ([4, 3-b]) indoles of formula (1) or formula (2) as means for improving cognitive functions and memory.

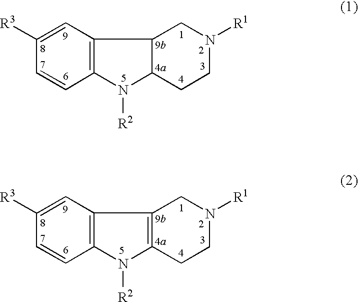

### CROSS-REFERENCE TO RELATED APPLICATIONS

This application claims priority to Russian Patent Application No. 2006142521, filed Dec. 1, 2006, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The invention relates to the use of chemical compounds in the field of medicine and may be utilized as a means for the manufacture of pharmacological preparations for improving cognitive functions and memory.

### BACKGROUND OF THE INVENTION

Impaired cognitive functions and memory loss are both a consequence of natural age-related changes (R. Levy (1994) “Aging-associated cognitive decline,” Int. Psychogeriatr. 6:63-68), and also occur as a result of various pathologies of the central nervous system, both acute (e.g., physical and psychic trauma, poisoning, hypoxia, stress, etc.) and chronic (e.g., neurodegenerative diseases, depression, alcoholism, neuroinfection, etc.). A special type of cognitive function decline has recently been identified, called “mild cognitive impairment,” (“MCI”) most commonly observed in the elderly and aged. MCI is characterized by a more pronounced deterioration in cognitive functions than is typical for normal age-related decline. The etiology of this illness is unknown and, apparently, is not directly related to neurodegenerative processes in the brain (S. I. Gavrilova, “The concept of mild cognitive decline,” in Alzheimer's disease and aging (Mater. III Ros. Konf. Moscow, Pul's) pp. 9-20). In addition, situations frequently arise in the course of human life which require significant activation of the cognitive functions and the need to remember a large volume of material.

As a rule, dementia with Alzheimer's disease or similar neurodegenerative conditions is defined as a persistent disorder of the cognitive functions (i.e., memory, etc.) accompanied by some type of structural or metabolic brain damage. That damage progresses over time, eventually leading to the inability to perform basic social and professional functions (desadaptation). Patients with mild cognitive impairment are not cognitively impaired to the same extent as patients suffering from Alzheimer's or other similar dementias. Furthermore, MCI patients have difficulty performing complex daily tasks and learning, in contrast to the cognitive impairment associated with Alzheimer's and other similar dementias, which is characterized by a inability to perform cognitive tasks relating to social, everyday, and/or professional functions (desadaptations).

A solution to the problem of improving human memory and cognitive functions has been sought worldwide for many years, but the mechanism of these, in the general case, has not so far been established. Medical practice has a small number of preparations which are somehow capable of improving memory. First and foremost among these is the relatively recently discovered class of what are termed nootropics—substances which are able to improve brain operation, have antihypoxic and general protective properties, and facilitate restoration of impaired thought functions and memory (M. D. Mashkovskiy (1998) Lekarstvennye sredstva, 1:108-116, Khar'kov). They are widely used in such pathological conditions as brain trauma, insult and chronic cerebrovascular insufficiency, post-hypoxic encephalopathy, neuroinfection, neuro-degenerative affection of the brain, chronic alcoholism, and delayed development in children. However, the nootropic preparations which are used at the present time are insufficiently effective in certain types of pathologies, or have high toxicity (T. A. Voronina (2003) Eksperim. Klinich. Farmakol. 66(2):10-14). For example, the preparation Nootropil® (piracetam), which is widely used in clinical practice had a stimulating effect on learning and memory in active and passive avoidance conditioned response tests only in doses of 200-500 mg/kg, depending on the test (R. U. Ostrovskaya, T. A. Gudasheva, T. A. Voronina, and S. B. Seredenin (2002) Exp. Clin. Pharmacol. 65(5):66-72). There are virtually no substances which would exert a rapid (e.g., within one hour), specific activating effect on cognitive functions and memory. For Nootropil® (piracetam) to manifest stimulating properties requires that it be used for several weeks or that enormous doses (in the range of hundreds of mg/kg) be used.

Certain known compounds, including derivatives of tetra- and hexa-hydro-1H-pyrido([4,3-b]-indole, exhibit a wide spectrum of biological activity. For example, in the series of 2,3,4,5-tetrahydro-1H-pyrido[4,3-b]indoles, the following types of activity have been observed: antihistamine (OS-DE No. 1813229 of 6 Dec. 1968, No. 1952800 of 20 Oct. 1969), central-depressive and anti-inflammatory activity (U.S. Pat. No. 3,718,657, issued 13 Dec. 1970), neuroleptic activity (C. A. Herbert, S. S. Plattner, and W. N. Welch (1980) Mol. Pharm., 17(1):38-42) and others. Derivatives of 2,3,4,4a,5,9b-hexahydro-1H-pyrido[4,3-b]indole exhibit psychotropic (W. N. Welch, C.A. Herbert, A. Weissman, and K. B. Koe (1986) J. Med. Chem. 29(10):2093-2099), antidepressive, antiarrhythmic and other types of activity.

As described in U.S. Pat. Nos. 6,187,785 (“the '785 patent”) and 7,021,206 (“the '206 patent”), hydrogenated pyrido[4,3-b]indole derivatives such as dimebon have NMDA antagonist properties, which make them useful for treating neurodegenerative diseases, such as Alzheimer's disease. Dimebon can be useful for treating Alzheimer's disease and other neurodegenerative diseases both alone (as described in the '785 patent and the '206 patent) and in combination with other compounds (as described in a PCT application claiming priority to U.S. Provisional Patent Application No. 60/854,866, filed Oct. 26, 2007). As described in WO 2005/055951, hydrogenated pyrido[4,3-b]indole derivatives, such as dimebon, are useful as human or veterinary geroprotectors, e.g., by delaying the onset and/or development of an age-associated or related manifestation and/or pathology or condition, including disturbance in skin-hair integument, vision disturbance and weight loss. As described in U.S. patent application Ser. Nos. 11/543,529 (U.S. Patent Publication No. 2007-0117835-A1) and 11/543,341 (U.S. Patent Publication No. 2007-0117834-A1), hydrogenated pyrido[4,3-b]indole derivatives such as dimebon are useful as neuroprotectors for use in treating and/or preventing and/or slowing the progression or onset and/or development of Huntington's disease. As described in WO 2007/087425, published Aug. 2, 2007, hydrogenated pyrido[4,3-b]indole derivatives such as dimebon are useful for treating schizophrenia. As described in WO 2007/020516, filed Sep. 20, 2007, hydrogenated pyrido[4,3-b]indole derivatives such as dimebon are useful for treating amyotrophic lateral sclerosis. Hydrogenated pyrido[4,3-b]indole derivatives are also useful for treatment of ischemic or hemorrhagic insult, as disclosed in a PCT application claiming priority to Russian Patent Application No. 2006143332.

There remains a significant medical need for additional or alternative therapies for treating mild cognitive impairment accompanied by memory loss. Preferably, the therapeutic agents can improve the memory, improve the quality of life, reduce impairment of cognitive function, and/or prolong the survival time for patients suffering from mild cognitive impairment.

The task, to the solution of which the invention now proposed is directed, is to extend the arsenal of means which can be utilized as new effective stimulators of cognitive functions and memory.

### DETAILED DESCRIPTION OF THE INVENTION

### Definitions

As used herein, unless clearly indicated otherwise, the terms “a,” “an,” and the like refer to one or more. It is also understood and clearly conveyed by this disclosure that reference to “the compound” or “a compound” includes and refers to any compound or pharmaceutically acceptable salt or other form thereof as described herein, such as the compound dimebon.

As used herein, the term “mild cognitive impairment” or “MCI” refers to a type of cognitive disorder characterized by a more pronounced deterioration in cognitive functions than is typical for normal age-related decline. As a result, elderly or aged patients with MCI have greater than normal difficulty performing complex daily tasks and learning, but without the inability to perform normal social, everyday, and/or professional functions typical of patients with Alzheimer's disease, or other similar neurodegenerative disorders eventually resulting in dementia. The etiology of this illness is unknown and, apparently, is not directly related to neurodegenerative processes in the brain (S. I. Gavrilova, “The concept of mild cognitive decline,” in Alzheimer's disease and aging (Mater. III Ros. Konf. Moscow, Pul's) pp. 9-20).

As used herein, unless clearly indicated otherwise, the term “an individual” refers to a mammal, including but not limited to a human. The individual may be a human who has been diagnosed with or is suspected of having mild cognitive impairment. The individual may be a human who exhibits one or more symptoms associated with mild cognitive impairment. The individual may be a human who has a mutated or abnormal gene associated with elevated risk of mild cognitive impairment but who has not been diagnosed with the disorder. The individual may be a human who is genetically or otherwise predisposed to developing mild cognitive impairment.

In one variation, the individual is a human who has not been diagnosed with and/or is not considered at risk for developing Alzheimer's disease, Huntington's disease, amyotrophic lateral sclerosis, or schizophrenia. In one variation, the individual is a human who does not have a non-life threatening condition associated with the aging process (such as loss of sight (cataract), deterioration of the dermatohairy integument (alopecia) or an age-associated decrease in weight due to the death of muscular and fatty cells) or a combination thereof. In one variation, the individual is a human who has not suffered an ischemic or hemorrhagic insult.

As used herein, an “at risk” individual is an individual who is at risk of developing or suffering mild cognitive impairment. An individual “at risk” may or may not have detectable disease, and may or may not have displayed detectable disease prior to the treatment methods described herein. “At risk” denotes that an individual has one or more so-called risk factors, which are measurable parameters that correlate with likelihood of developing or experiencing mild cognitive impairment. An individual having one or more of these risk factors has a higher probability of developing the disorder than an individual without those risk factor(s). Risk factors include, but are not limited to, age, sex, race, diet, history of previous disease, presence of precursor disease, genetic (i.e., hereditary) considerations, and environmental exposure. Individuals at risk for mild cognitive impairment include, e.g., those having relatives who have experienced MCI, and those whose risk is determined by analysis of genetic or biochemical markers.

As used herein, the term “pharmaceutically active compound,” “pharmacologically active compound” or “active ingredient” refers to a chemical compound, such as a hydrogenated pyrido (4,3-b) indole, that induces a desired effect, e.g., treating and/or preventing and/or delaying the onset of mild cognitive impairment.

As used herein, the term “pharmacological means” or “pharmaceutical formulation” refers to the use of any therapeutic dosage form, including immediate or sustained release forms, containing a compound of the invention, e.g., of formula (1) or formula (2), which may find prophylactic or therapeutic use in medicine for the treatment of mild cognitive impairment. Such means or formulations may contain may also contain pharmaceutically acceptable excipients, including preservatives, solubilizers, stabilizers, re-wetting agents, emulgators, sweeteners, dyes, adjusters, salts for the adjustment of osmotic pressure, buffers, coating agents or antioxidants.

As used herein, the term “pharmaceutically acceptable” or “pharmacologically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “effective amount” refers to the use of that amount of compounds of the invention, e.g., of formula (1) or formula (2) which in combination with its activity and toxicity characteristics, and also on the basis of the knowledge of a specialist, should be effective in a given therapeutic form.

As used herein, the term “therapeutically effective amount” refers to an amount of a compound or a combination therapy sufficient to produce a desired therapeutic outcome (e.g., reducing the severity or duration of, stabilizing the severity of, or eliminating one or more symptoms associated with mild cognitive impairment). For therapeutic use, beneficial or desired results include, e.g., improving cognition or otherwise reversing cognitive impairment, improving memory or otherwise reversing loss of memory, decreasing one or more symptoms resulting from the disease (biochemical, histologic and/or behavioral), including associated complications and intermediate pathological phenotypes presenting during development or progression of mild cognitive impairment, increasing the quality of life of those suffering mild cognitive impairment, decreasing the dose of other medications required to treat the mild cognitive impairment, enhancing effect of another medication, and/or prolonging survival of patients.

A “prophylactically effective amount” refers to an amount of a compound or a combination therapy sufficient to prevent or reduce the severity of one or more future symptoms of mild cognitive impairment when administered to an individual who is susceptible and/or who may develop such impairment. For prophylactic use, beneficial or desired results include, e.g., results such as eliminating or reducing the risk, lessening the severity, or delaying the onset of mild cognitive impairment, including biochemical, histologic and/or behavioral symptoms of mild cognitive impairment, its complications and intermediate pathological phenotypes presenting during development and/or progression of the disease.

As used herein, “treatment” or “treating” is an approach for obtaining beneficial or desired results, including clinical results. For purposes of this invention, beneficial or desired clinical results include, but are not limited to, one or more of the following: decreasing one more symptoms resulting from mild cognitive impairment, limiting the extent of disability resulting from mild cognitive impairment, increasing the quality of life, reducing any impairment of cognitive function, improving memory and/or cognition, decreasing the dose of one or more other medications required to treat the disease, and/or prolonging survival time for individuals suffering from mild cognitive impairment. In some embodiments, an individual or combination therapy of the invention reduces the severity of one or more symptoms associated with mild cognitive impairment by at least 10, 20, 30, 40, 50, 60, 70, 80, 90, or 95% compared to the corresponding symptom in the same subject prior to treatment or compared to the corresponding symptom in other subjects not receiving the therapy.

As used herein, the term “combination therapy” refers to a first therapy that includes one or more hydrogenated pyrido[4,3-b]indoles or pharmaceutically acceptable salts thereof in conjunction with a second therapy that includes one or more other compounds (or pharmaceutically acceptable salts thereof) or therapies (e.g., surgical procedures) useful for decreasing one more symptoms resulting from mild cognitive impairment, limiting the extent of disability resulting from mild cognitive impairment, increasing the quality of life, reducing any impairment of cognitive function, decreasing the dose of one or more other medications required to treat the disease, and/or prolonging survival time for individuals suffering from mild cognitive impairment. Administration in “conjunction with” another compound includes administration in the same or different composition, either sequentially, simultaneously, or continuously using the same or different route of administration for each compound. In some variations, the combination therapy optionally includes one or more pharmaceutically acceptable carriers or excipients, non-pharmaceutically active compounds, and/or inert substances.

As used herein, the term “simultaneous administration” means that a first therapy and a second therapy of a combination therapy are administered with a time separation of no more than about 15 minutes, such as no more than about any of 10, 5, or 1 minutes. When the compounds are administered simultaneously, the first and second therapies may be contained in the same composition (e.g., a composition comprising both a hydrogenated pyrido[4,3-b]indole and the nootropic agent Memantine®) or in separate compositions (e.g., a hydrogenated pyrido[4,3-b]indole is contained in one composition and Memantine® is contained in another composition).

As used herein, the term “sequential administration” means that the first therapy and second therapy in a combination therapy are administered with a time separation of more than about 15 minutes, such as more than about any of 20, 30, 40, 50, 60 or more minutes. Either therapy may be administered first. The first and second therapies are contained in separate compositions, which may be contained in the same or different packages or kits.

Thus, an effective amount of a combination therapy includes an amount of the first therapy and an amount of the second therapy that when administered sequentially, simultaneously, or continuously produces a desired outcome. Suitable doses of any of the co-administered compounds may optionally be lowered due to the combined action (e.g., additive or synergistic effects) of the compounds. In various embodiments, treatment with the combination of the first and second therapies may result in an additive or even synergistic (e.g., greater than additive) result compared to administration of either therapy alone. In some embodiments, a lower amount of each pharmaceutically active compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. Preferably, the use of a small amount of pharmaceutically active compound results in a reduction in the number, severity, frequency, or duration of one or more side-effects associated with the compound.

As is understood in the clinical context, an effective dosage of a drug, compound or pharmaceutical composition containing a compound described by formula (1) or (2) or any compound described herein (e.g., any of compounds 1 to 9) may be achieved in conjunction with another drug, compound or pharmaceutical composition that contains one or more compounds that improve brain function, that have antihypoxic or general protective properties, that facilitate restoration of impaired cognitive functions and memory (e.g., nootropic agents such as Memantine® or piracetam), that antagonize NMDA receptors (e.g., Memantine® (also available as Namenda®, Axura®, Akatinol®, and Ebixa®), meramexane, amantadine, dextrorphan, ketamine, and the like), and that inhibit cholinesterase activity (e.g., Aricept® (Donepezil HCl) and physostigmine).

As used herein, the term “controlled release,” “sustained release,” or “delayed release” refers to a drug-containing formulation or fraction thereof in which release of the drug is not immediate, i.e., with a “controlled,” “sustained,” or “delayed release” formulation, administration does not result in immediate release of the drug into an absorption pool. In certain embodiments, the compound is administered to the individual as a sustained release form or as part of a sustained release system, such as a system capable of sustaining the rate of delivery of a compound to an individual for a desired duration, which may be an extended duration such as a duration that is longer than the time required for a corresponding immediate-release dosage form to release the same amount (e.g., by weight or by moles) of compound, and can be hours or days. A desired duration may be at least the drug elimination half-life of the administered compound and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

### Exemplary Hydrogenated Pyrido (4,3-b) Indoles

Hydrogenated pyrido[4,3-b]indoles of formula (1) or formula (2) can be used to improve cognitive functions and memory.

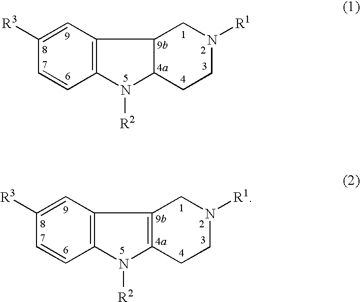

When compounds of formula (1) are used, R¹ is selected from the group containing CH₃—, CH₃CH₂— or PhCH₂; R² is selected from the group containing H, PhCH₂ or 6-CH₃-Py-(CH₂)₂—; and R³ is selected from the group containing H, CH₃— or Br—. Those compounds may comprise salts with pharmaceutically acceptable acids.

One of the compounds which may be used as a means for improving cognitive functions and memory may be compounds of formula (1) in which R¹ corresponds to CH₃—; R² is H—; and R³ is CH₃—. When compounds of formula (2) are used, R¹ is selected from the group containing CH₃—, CH₃CH₂— or PhCH₂—; R² is selected from the group containing H—, PhCH₂— or 6-CH₃-3-Py—(CH₂)₂—; and R³ is selected from the group containing H—, CH₃— or Br—. Those compounds may comprise salts with pharmaceutically acceptable acids.

One of the compounds which may be used as a means for improving cognitive functions and memory may be compounds of formula (2) in which R¹ corresponds to CH₃CH₂— or PhCH₂—; R² corresponds to H—; and R³ is H—; or a compound where R¹ corresponds to CH₃—; R² corresponds to PhCH₂—; and R³ is CH₃—; or a compound where R¹ corresponds to CH₃—; R² corresponds to 6-CH₃-3-Py—(CH₂)₂—; and R³ is H—; or a compound where R¹ corresponds to CH₃—; R² corresponds to 6-CH₃-3-Py—(CH₂)₂—; and R³ is CH₃—; or a compound where R¹ corresponds to CH₃—, R² corresponds to H—; and R³ is H— or CH₃—; or a compound where R¹ corresponds to CH₃—, R² corresponds to H—, and R³ is Br—.

Any of the compounds indicated above may be used as a means for improving cognitive functions and memory.

Known compounds of formula (1) and (2) are widely used in pharmacological practice and include the following specific compounds:
1. cis(±)2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]-indole;
4. 2,8-dimethyl-5-benzyl-2,3,4,5-tetrahydro-1H-pyrido[4,3-b]indole and its hydrochloride;
5. 2-methyl-5-[2-(6-methyl-3-pyridyl)ethyl]-2,3,4,5-tetrahydro-1H-pyrido[4,3-b]indole and its sesquisulfate monohydrate;
6. 2,8-dimethyl-5-[2-(6-methyl-3-pyridyl)ethyl]-2,3,4,5-tetrahydro-1H-pyrido[4,3-b]indole and its dihydrochloride (dimebon);
7. 2-methyl-2,3,4,5-tetrahydro-1H-pyrido[4,3-b]-indole;
8. 2,8-dimethyl-2,3,4,5-tetrahydro-1H-pyrido[4,3-b]-indole and its methyl iodide;
9. 2-methyl-8-bromo-2,3,4,5-tetrahydro-1H-pyrido-4,3-b]indole and its hydrochloride.

Extensive investigations have been carried out into a series of known compounds which comprise derivatives of tetra- and hexa-hydro-1H-pyrido[4,3-b]-indole and which exhibit a wide spectrum of biological activity. The following types of activity were found in the 2,3,4,5-tetrahydro-1H-pyrido[4,3-b]indole series: antihistamine (OS-DE No. 1813229 of 6 Dec. 1968, No. 1952800 of 20 Oct. 1969), central-depressant, anti-inflammatory (U.S. Pat. No. 3,718,657, issued 13 Dec. 1970), neuroleptic (C. A. Herbert, S. S. Plattner, and W. N. Welch (1980) Mol. Pharm. 17(1):38-42) and others. Derivatives of 2,3,4,4a,5,9b-hexahydro-1H-pyrido[4,3-b]indole exhibit psychotropic (W. H. Welch, C. A. Herbert, A. Weissman and K. B. Koe (1986) J. Med. Chem. 29(10):2093-2099), antidepressive, antiarythmic and other types of activity.

A number of therapeutic preparations based on derivatives of tetra- and hexa-hydro-1H-pyrido[4,3-b]-indole are manufactured: diazoline (mebhydroline), dimebon, dorastine, carbidine (dicarbine), stobadine, hevotroline. Diazoline (2-methyl-5-benzyl-2,3,4,5-tetrahydro-1H-pyrido[4,3-b]indole) dihydrochloride (M. A. Klyuev (1991) Drugs used in USSR medical practice (Moscow, Meditsina) p. 512) and dimebon (2,8-dimethyl-5-(2-(6-methyl-pyridyl-3)ethyl)-2,3,4,5-tetra-hydro-1H-pyrido[4,3-b]indole) (M. D. Mashkovskiy (1993) Medicinal drugs in 2 parts, (12th ed. Moscow, Meditsina) Part 1, p. 383), and also its close analog dorastine (2-methyl-8-chloro-5-(2-(6-methyl-3-pyridyl)-ethyl)-2,3,4,5-tetrahydro-1H-pyrido[4,3-b]indole)dihydrochloride (USAN and USP dictionary of drugs names (United States Adopted Names 1961-1988, current U.S. Pharmacopeia and National Formulary for Drugs, and other nonproprietary drug names (1989) 26th ed., p. 196) are known as antihistamine preparations. Carbidine (dicarbine) (cis(±)2,8-dimethyl-2,3,4,4a,5,9b-hexahydro-1H-pyrido[4,3-b]indole dihydro-chloride) is a Russian produced neuroleptic with an antidepressive effect (L. N. Yakhontov and R. G. Glushkov (1983) Synthetic medicinal drugs (Ed. A. G. Natradze, Moscow, Meditsina) pp. 234-237), while its (−)-isomer, stobadine, is known as an anti-arrhythmia drug (M. Kitlova, P. Gibela, and J. Drimal (1985) Bratisl. Lek. Listy 84(5):542-546); hevotroline (8-fluoro-2-(3-3-pyridyl)propyl)-2,3,4,5-tetrahydro-1H-pyrido[4,3-b]indole) is an antipsychotic and anxiolytic drug (M. Abou-Gharbi, U. R. Patel, M. B. Webb, J. A. Moyer, and T. H. Ardnee (1987) J. Med. Chem. 30:1818-1823).

It has previously been demonstrated that dimebon exhibits clearly marked properties of antagonists of NMDA receptors, allowing cognitive functions and memory to be restored in animals with a model of Alzheimer's disease, induced by chronic administration of the cholinotoxin AF64A. After receiving dimebon, rats with a model of AD, in which the cholinergic neurons had first been damaged, demonstrated significantly better results in an active avoidance conditioned reflex experiment than control animals which had not received dimebon (S. Bachurin, E. Bukatina, N. Lermontova et al. (2001) Ann. N.Y. Acad. Sci. 939:425-435). Furthermore, dimebon has a favorable effect on patients with Alzheimer's disease. Modern ideas about the mechanisms of pathogenesis of Alzheimer's disease (AD) speak of the existence of numerous neurotic plaques on the surface of a number of areas of the brain. The β-amyloid protein found in them has a neurotoxic effect and causes the death of primarily cholinergic neurons. Cholinergic preparations, cholinesterase inhibitors such as aricept, physostigmine and others, are thus widely used for the treatment of AD. The death of neurons is associated with deterioration of memory and cognitive functions and with other neurodegenerative disorders (see, e.g., Alzheimer's Disease and Related Disorders (1999) (Wiley & Sons, Ed. K. Iqbal et al.) pp. 1-819). In pilot clinical studies, it has been shown that the use of dimebon in a dose of 20 mg three times daily provides improvement in the condition of AD patients having pronounced memory disorder, i.e. restoration of memory and cognitive functions (RF patent No. 2106864).

In our studies, it was found unexpectedly that the use of hydrogenated pyrido[4,3-b]indoles of formula (1) or formula (2) produces a significant improvement in memory and cognitive functions, not only in the presence of neurodegenerative diseases, but also in animals in which the destruction of neurons has not occurred, i.e., not associated with the presence in them of the previously known properties of antagonists of NMDA receptors.

The deterioration in memory and cognitive functions which is observed in the elderly and aged, and in brain trauma, depression, alcoholism, neuroinfections, stress, excessive fatigue, mild cognitive impairment, etc., is not associated with the death of neurons (and, in particular, cholinergic neurons), but infers other mechanisms. These include: deterioration in the supply of oxygen and glucose to brain neurons not associated with neuronal death, disturbance of neuronal links, including between the cortex and the subcortical structures, the action of neurotoxins, disturbance of the synthesis of proteins and nucleic acids, hindering of the appearance of new neurons in the brain (S. Rouz (1995) Ustroystvo pamyati (Moscow, Mir); D. L. Schacter (1996) Searching for Memory: The Brain, the Mind and the Past (Basic Books); G. Edelman (1989) The Remembered Present: A Biological Theory of Consciousness (Basic Books)).

According to the invention, a pharmacological means for the treatment of mild cognitive impairment, containing an active principle and a pharmaceutically acceptable carrier, contains as the active principle an effective amount of a hydrogenated pyrido(4,3-b)indole of formula (1) or formula (2).

In order to prepare a pharmacological means, one or several compounds of formula (1) or formula (2) are mixed as the active ingredient with a pharmaceutically acceptable carrier, known in medicine, in accordance with methods adopted in pharmaceuticals. The carrier may have various forms, depending on the therapeutic form of the preparation.

In accordance with the invention, a method for the treatment of mild cognitive impairment comprises administering to a patient a pharmacological means containing an effective amount of a hydrogenated pyrido(4,3-b) indole of formula (1) or formula (2), such as dimebon, in a dose of 0.01-10 mg/kg of body weight at least once daily for a period necessary to achieve a therapeutic effect. The invention further provides methods for the treatment of mild cognitive impairment comprising administering to a patient a pharmaceutical means containing an effective amount of a hydrogenated pyrido (4,3-b) indole of formula (1) or formula (2), wherein the hydrogenated pyrido (4,3-b) indole is compound 1, compound 2, compound 3, compound 4, compound 5, compound 6, compound 7, compound 8, or compound 9, or a pharmaceutically acceptable salt thereof, in a dose of 0.01-10 mg/kg of body weight at least once daily for a period necessary to achieve a therapeutic effect. In certain embodiments, the pharmaceutical means is administered intravenously at doses ranging from 0.15 to 0.3 mg/kg one or more times daily for a period necessary to achieve a therapeutic effect. In certain embodiments, the pharmaceutical means is administered orally in doses of 5-20 mg from one to three times daily for a period necessary to achieve a therapeutic effect.

In certain embodiments, the pharmaceutical means containing an effective amount of a hydrogenated pyrido(4,3-b) indole of formula (1) or formula (2), such as dimebon, is administered in combination with a second therapy that includes one or more other compounds (or pharmaceutically acceptable salts thereof) or therapies (e.g., surgical procedures) useful for decreasing one more symptoms resulting from mild cognitive impairment, limiting the extent of disability resulting from mild cognitive impairment, increasing the quality of life, reducing any impairment of cognitive function, decreasing the dose of one or more other medications required to treat the disease, and/or prolonging survival time for individuals suffering from mild cognitive impairment.

In certain embodiments, the pharmaceutical means containing an effective amount of a hydrogenated pyrido(4,3-b) indole of formula (1) or formula (2), wherein the hydrogenated pyrido (4,3-b) indole is compound 1, compound 2, compound 3, compound 4, compound 5, compound 6, compound 7, compound 8, or compound 9, or a pharmaceutically acceptable salt thereof, is administered in combination with a second therapy that includes one or more other compounds (or pharmaceutically acceptable salts thereof) or therapies (e.g., surgical procedures) useful for decreasing one more symptoms resulting from mild cognitive impairment, limiting the extent of disability resulting from mild cognitive impairment, increasing the quality of life, reducing any impairment of cognitive function, decreasing the dose of one or more other medications required to treat the disease, and/or prolonging survival time for individuals suffering from mild cognitive impairment.

### Exemplary Formulations

One or more compounds of formula (1) or formula (2) can be used in the preparation of a formulation, such as a pharmaceutical formulation, by combining the compound or compounds as active ingredient with a pharmaceutically acceptable carrier, which are known in the art. See, e.g., Remington's Pharmaceutical Sciences, 20th ed. (2000), Mack Publishing Co., Philadelphia, Pa., which is incorporated herein by reference. Depending on the therapeutic form of the system (e.g., intravenous injection versus oral tablet), the carrier may be in various forms.

Pharmaceutical formulations may be administered in the form of conventional oral compositions, such as tablets, coated tablets, gelatin capsules with hard and soft coating, emulsions or suspensions. Preferably, however, they have liquid forms, suitable for intravenous injections or for droppers. Examples of carriers which can be utilized for the manufacture of such compositions are lactose, maize starch or its derivatives, talc, stearic acid or its salts, etc. Acceptable carriers for gelatin capsules with a soft coating are, for example, vegetable oils, waxes, fats, semi-solid and liquid polyols, etc. In addition, pharmaceutical preparations may contain preservatives, solubilizers, stabilizers, wetting agents, emulsifiers, sweeteners, colorants, correctives, salts for altering osmotic pressure, buffers, coating agents or antioxidants. They may also contain other substances which have desirable therapeutic properties. Preparative forms may comprise the normal standard dose and may be prepared by methods well known in pharmacy. Suitable formulations can be found, e.g., in Remington's Pharmaceutical Sciences, supra, which is incorporated herein by reference.

### Exemplary Dosing Regimens

For use herein, unless clearly indicated otherwise, a compound or combination therapy of the invention may be administered to the individual by any available dosage form. In one variation, the compound or combination therapy is administered to the individual as a conventional immediate release dosage form. In one variation, the compound or combination therapy is administered to the individual as a sustained release form or part of a sustained release system, such as a system capable of sustaining the rate of delivery of a compound to an individual for a desired duration, which may be an extended duration, such as a duration that is longer than the time required for a corresponding immediate-release dosage form to release the same amount (e.g., by weight or by moles) of compound or combination therapy, and can be hours or days. A desired duration may be at least the drug elimination half life of the administered compound or combination therapy and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The compound or combination therapy may be formulated for any available delivery route, whether immediate or sustained release, including an oral, mucosal (e.g., nasal, sublingual, vaginal, buccal or rectal), parenteral (e.g., intramuscular, subcutaneous, or intravenous), topical or transdermal delivery form. A compound or combination therapy may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

The amount of compound, such as dimebon or any of compounds 1 to 9, in a delivery form may be any effective amount, which may be from about 10 ng to about 1,500 mg or more of the single active ingredient compound of a monotherapy or of more than one active ingredient compound of a combination therapy. In one variation, a delivery form, such as a sustained release system, comprises less than about 30 mg of compound. In one variation, a delivery form, such as a single sustained release system capable of multi-day administration, comprises an amount of compound such that the daily dose of compound is less than about 30 mg of compound.

A treatment regimen involving a dosage form of compound, whether immediate release or a sustained release system, may involve administering the compound to the individual in dose of between about 0.1 and about 10 mg/kg of body weight, at least once a day and during the period of time required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered, such as a daily dosage that is less than about 0.1 mg/kg, which may include but is not limited to, a daily dosage of about 0.05 mg/kg.

The compound, such as dimebon or any of compounds 1 to 9, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosing frequency can be about a once weekly dosing. The dosing frequency can be about a once daily dosing. The dosing frequency can be more than about once weekly dosing. The dosing frequency can be less than three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more). The dosing frequency can be continuous (e.g., once weekly dosing for continuous weeks). Any of the dosing frequencies can employ any of the compounds described herein together with any of the dosages described herein, for example, the dosing frequency can be a once daily dosage of less than 0.1 mg/kg or less than about 0.05 mg/kg of dimebon.

In one variation, dimebon is administered in a dose of 5 mg once a day. In one variation, dimebon is administered in a dose of 5 mg twice a day. In one variation, dimebon is administered in a dose of 5 mg three times a day. In one variation, dimebon is administered in a dose of 10 mg once a day. In one variation, dimebon is administered in a dose of 10 mg twice a day. In one variation, dimebon is administered in a dose of 10 mg three times a day. In one variation, dimebon is administered in a dose of 20 mg once a day. In one variation, dimebon is administered in a dose of 20 mg twice a day. In one variation, dimebon is administered in a dose of 20 mg three times a day. In one variation, dimebon is administered in a dose of 40 mg once a day. In one variation, dimebon is administered in a dose of 40 mg twice a day. In one variation, dimebon is administered in a dose of 40 mg three times a day.

### Exemplary Kits

The invention further provides kits comprising one or more compounds as described herein. The kits may employ any of the compounds disclosed herein and instructions for use. In one variation, the kit employs dimebon. In other variations, the kit comprises one or more of compounds 1 to 9. The compound may be formulated in any acceptable form. The kits may be used for any one or more of the uses described herein, and, accordingly, may contain instructions for any one or more of the stated uses (e.g., decreasing one more symptoms associated with mild cognitive impairment, limiting the extent of disability resulting from mild cognitive impairment, increasing the quality of life, and/or reducing any impairment of cognitive function).

Kits generally comprise suitable packaging. The kits may comprise one or more containers comprising any compound described herein, in unit dosage form or in multiple dosage form. Each component (if there is more than one component) can be packaged in separate containers or some components can be combined in one container where cross-reactivity and shelf life permit. The kit components can be supplied as liquids or powders. If supplied as powders, the kits may further comprise a pharmaceutically acceptable buffer or other solution for preparing a liquid formulation of the compound.

The kits may optionally include instructions, generally written instructions, although electronic storage media (e.g., magnetic diskette or optical disk) containing instructions are also acceptable, relating to the use of component(s) of the kit in methods of the present invention (e.g., methods of treating mild cognitive impairment). The instructions included with the kit generally include, for example, information describing the components of the kit and methods of administering those components to an individual in need thereof.

The technical results which can be secured when implementing the invention include improvement in cognitive functions and memory in the elderly and aged, in mild cognitive impairment, and in brain trauma, depression, alcoholism, neuroinfections, stress, excessive fatigue and developmental arrest in children (i.e., pathologies not associated with the death of neurons and, in particular, cholinergic neurons).

The possibility of implementing the invention with achievement of the stated purpose is confirmed, but not exhausted, by the following findings.

### EXAMPLES

### Example 1

A behavioral experiment with the object of elucidating the action on cognitive functions and memory in experimental animals of a representative of the hydrogenated pyrido([4,3-b])indoles of formula (1) or formula (2)-dimebon.

Method. In order to study the action of substances on the memory of animals in which there had been no prior destruction of neurons, we used the test of recognition of the new location of a known object (“Object location memory test”, B. Kolb, K. Buhrmann, R. McDonald and R. Sutherland, Cereb. Cortex, 6 (1994), pp 664-680; D. Gaffan, Eur. J. Neurosci., 4 (1992), pp. 381-388; T. Steckler, W. H. I. M. Drinkenburgh, A. Sahgal and J. P. Aggleton, Prog. Neurobiol., 54 (1998), pp. 289-311). This Object Recognition Test is used to study memory. It provides a reliable, easy-to-use assessment tool for analyzing the effects of test compounds on memory in animals. The test can be applied to normal healthy animals (controls), as well as to animals that are models of various neurodegenerative diseases.

Experiments were performed on healthy, mature C57BL/6 male mice aged 3-5 months and weighing 20-24 g. Male mice were used to exclude any possible negative effects resulting from hormonal changes associated with menstruation in female mice. The animals were kept in a vivarium with 5 to a cage in 12/12 hours light regime with light from 08.00 to 20.00 and free access to water and food. The observation chamber was made from white opaque organic glass and measured 48×38×30 cm. Brown glass vials with a diameter of 2.7 cm and a height of 5.5 cm were used as the test objects. 2-3 minutes before introducing an animal, the chamber and test objects were rubbed with 85% alcohol. The animals were always placed in the centre of the chamber.

Dimebon was dissolved in distilled water and administered intragastrically 1 hour before training in a volume of 0.05 ml per 10 g of animal weight. A corresponding volume of solvent was administered to control animals.

Procedure for Performing the Behavioral Experiment.

On the first day, the mice were brought into the test room and acclimatized for 20-30 minutes. After this, each animal was placed for 10 min. in an empty behavior chamber, which had been pretreated with alcohol, for familiarization. The animal was then replaced in the cage and taken to the vivarium.

On the following day, the same mice were brought into the test room, acclimatized for 20-30 min. and then given dimebon solution intragastrically. One hour after administration of the substance, an animal was placed in the behavior chamber, on the bottom of which two identical objects for recognition (glass vials) were placed on a diagonal at a distance of 14.5 cm from the corners. The training time for each animal was 20 min. After 20 min. it was replaced in the cage and returned to the vivarium.

Testing was performed 48 hours after training. For this purpose, after acclimatization an animal was placed for 1 min. in the chamber for refamiliarization. After a minute it was removed and one object was placed on the bottom of the chamber in a location known to the animal, and the other in a new location. The time spent investigating each object separately over a period of 10 min. was recorded with an accuracy of 0.1 s using two electronic stopwatches. The behavior of the animals was observed through a mirror. Purposeful approach of an animal's nose towards an object at a distance of 2 cm or direct touching of an object with the nose was regarded as a positive investigative reaction.

Since significant variations in object investigation time between animals are observed in this test, we calculated the percent investigation time for each mouse using the formula tNl/(tKl+tNl)×100. The total time spent on investigation of the two objects was taken as 100%. The results were further processed using the Student t-test method.

Results. As a result of the experiments performed, it was established that when testing 48 hours after training the mice in the control group investigate the object in the known location for 46.9±7.9%, and that in the new location for 53.1±7.9% of the time (P=0.3), i.e. they accept the objects in both locations as new. However, the animals which had been given dimebon in a dose of 0.01 mg/kg spent 57.5±4.8% of the time on investigation of the object in the new location, and 42.5±4.8% of the time on that in the known location (P=0.06). The maximally expressed stimulating effect was observed after administration of dimebon in a dose of 0.1 mg/kg (FIG. 1). In this group, the mice investigated the object in the new location for 62.4±6.5%, and that in the known location for 37.6%, of the time (P=0.002). When the dose is increased to 0.25 mg/kg, its activating effect on memory disappears.

In contrast to dimebon, the comparison preparation Memantine®, which at the present time is widely used in clinical practice to treat disorders of mnestic and cognitive functions of varying origin, exerted a maximally expressed activating effect on memory in a dose of 2.5 mg/kg. It was demonstrated that in this group the mice spent 59.4±6.9% of the time on investigation of the object in a new location, and 40.6±6.9% on the object in a known location (P=0.003) (FIG. 2).

Similar results were obtained in animals treated with Dimebon, but at 0.01 mg/kg, a much lower dose than that required to produce the same effect with Memantine®. Dimebon-treated mice (0.01 mg/kg) spent 57.5±4.8% of the total time to locate the object in its new position, and 42.5±4.8% in its original position (P=0.0006). Therefore, administration of Memantine® at a dose of 2.5 mg/kg produces essentially the same effect on memory as administration of Dimebon at a dose of 0.01 mg/kg, suggesting that the memory-enhancing activity of Dimebon is 250 times higher than that of Memantine®. However, the maximum effect of Dimebon was observed at a dose of 0.1 mg/kg, whereas the same effect was observed of Memantine® only at a dose of 2.5 mg/kg. Thus, comparing the maximally effective dose of Memantine® to the maximally effective dose of Dimebon, the memory-enhancing activity of Dimebon is 25 times higher than that of Memantine®.

The memory-enhancing effect of Nootropil® (piracetam) was not assessed experimentally. Instead, data obtained from the literature was used, indicating that the maximally effective dose of Nootropil® (piracetam) that stimulated learning and memory was between 200-500 mg/kg. See, e.g., R. U. Ostrovskaya, T. A. Gadasheva, T. A. Voronina, and S. B. Seredenin (2002) “The novel nootropic and neuroprotector drug noopept (GVS-111),” Exp. Clin. Pharmacol. 65(5):66-72 (available in Russian).

Animals in the control group 48 hours after the training exercise spent the same amount of time locating the object in its new position as they did locating the object in its known position.

On the basis of the results presented, it can be concluded that, in regard to the stimulating effect on the memory of animals, dimebon is 25-250 times more active than Memantine® and 2000-5000 times more active than Nootropil® (piracetam).

On investigating the acute toxicity of dimebon, it was found that the LD₅₀ for mice on intragastric administration is more than 1000 mg/kg. Investigation of the acute toxicity of Memantine® established that the death of the animals is observed after its intragastric administration in a dose of 300 mg/kg. The LD₅₀ for mice on intragastric administration of Nootropil® (piracetam) is 8000 mg/kg.

The next aspect of the invention is a pharmacological means for improving cognitive functions and memory, containing an active principle and a pharmaceutically acceptable carrier, in which the active principle is an effective amount of a hydrogenated pyrido(4,3-b)indole of formula (1) or formula (2).

A pharmacological means according to the invention is prepared using procedures which are conventional in this field and includes a pharmacologically effective amount of an active agent comprising compounds of formula (1) and (2) (hereinafter referred to as “the active principle”), normally constituting from 1 to 20 wt. % or from 1 mg to 20 mg in a dose form, which consists of a tablet, granule, spheroid, bead, pill or capsule, in combination with one or more pharmaceutically acceptable auxiliary additives such as fillers, binders, disintegrants, adsorbents, aroma substances and flavoring agents. In accordance with known methods, pharmaceutical compositions may be presented in various liquid or solid forms.

Examples of solid medicinal forms include, for example, tablets, pills, gelatin capsules, etc.

Compositions are, as a rule, produced using standard procedures, which provide for mixing the active principle with a liquid or finely ground solid carrier.

Compositions according to the invention in the form of tablets contain from 1 to 20% of active principle and a filler (fillers) and/or a carrier (carriers). The following are used for tablets: a) fillers: beet sugar, lactose, glucose, sodium chloride, sorbitol, mannitol, glycol, disubstituted calcium phosphate; b) binders: aluminum magnesium silicate, starch paste, gelatin, tragacanth, methyl cellulose, carboxymethylcellulose and polyvinylpyrrolidone; c) disintegrants: dextrose, agar, alginic acid or its salts, starch, tween.

### Example 1

100 mg tablets, each containing 5 mg of Dimebon

| | |
|---|---|
| Dimebon | 5 mg |
| Lactose | 50.0 mg |
| Alginic acid | 20.0 mg |
| Citric acid | 5.0 mg |
| Tragacanth | 20.0 mg |

A tablet may be formed by pressing or molding of the active ingredient with one or more auxiliary ingredients.

Pressed tablets are prepared in a special unit. The active ingredient in free form, such as powder or granules, in an amount of 50 g (the amount of the substance needed to prepare 10000 tablets) is stirred with tragacanth as binder (200 g), mixed with lactose (550 g) as filler, and alginic acid (200 g) as disintegrant and citric acid (50 g) as odorant are added to the mixture.

Colorants and stabilizers are additionally used for gelatin capsules. Tetrazine and indigo are used as colorants; stabilizers may include sodium metabisulfite and sodium benzoate. The gelatin capsules now proposed contain from 1 to 20% of the active ingredient.

### Example 2

500 mg capsules, each containing 20 mg of Dimebon

| | |
|---|---|
| Compound 1 | 20 mg |
| Glycerol | 100.0 mg |
| Sugar syrup | 319.0 mg |
| Mint oil | 40.0 mg |
| Sodium benzoate | 10.0 mg |
| Ascorbic acid | 5.0 mg |
| Tetrazine | 5.0 mg |

200 g of the active substance (Dimebon) (the amount needed for the preparation of 10000 capsules) are finely ground and mixed in a mixer with glycerol (1000 g) and sugar syrup (3190 g). After stirring, mint oil (400 g), sodium benzoate (100 g), ascorbic acid (50 g) and tetrazine (50 g) are added to the mixture. Gelatin capsules are made by the drop method. This method makes it possible simultaneously to carry out dropwise metering of the solution of medicinal substance and heated gelatin mass (900 g of gelatin) into chilled vaseline oil. As a result, seamless spherical gelatin capsules filled with a medicinal mixture are formed, fully ready for use and containing 20 mg of active principle.

According to the invention, a method for improving cognitive functions and memory is comprised in the administration to a patient of a pharmacological means containing an effective amount of hydrogenated pyrido(4,3-b)indoles of formula (1) or formula (2), in a dose of 1-150 mg at least once daily for a period necessary to achieve a therapeutic effect.

The dose of active component—compounds of formula (1) or (2)—prescribed varies in dependence on many factors, such as the age, sex, and weight of a person, the specific compound prescribed, the method of administration and the form of the preparation in which the active compound is prescribed.

1. A method of treating mild cognitive impairment in an individual in need thereof comprising administering to the individual a therapeutically effective amount of hydrogenated pyrido(4,3-b)indole of the formula (1), or a pharmaceutically acceptable salt thereof, 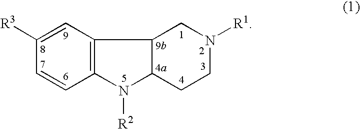
wherein:
R¹ is CH₃—, CH_{c}CH₂— or PhCH₂;
R² is H—, PhCH₂— or 6-CH₃-3-Py-(CH₂)₂—; and
R³ is H—, CH₃— or Br—.

2. The method of claim 1, wherein R¹ is CH₃—, R² is H—, and R³ is CH₃—.

3. The method of claim 2, wherein the compound is in the form of the (±) cis-isomer.

4. The method of claim 1, wherein the pharmaceutically acceptable salt is a pharmaceutically acceptable acid salt.

5. A method of treating mild cognitive impairment in an individual in need thereof comprising administering to the individual a therapeutically effective amount of hydrogenated pyrido(4,3-b)indole of the formula (2), or a pharmaceutically acceptable salt thereof, 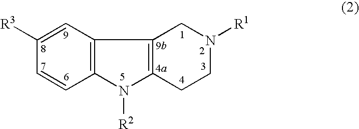
wherein:
R¹ is CH₃—, CH₃CH₂— or PhCH₃;
R² is H—, PhCH₂— or 6-CH₃-3-Py-(CH₂)₂—, and
R³ is H—, CH₃— or Br—.

6. The method of claim 5, wherein R¹ is CH₃CH₂— or PhCH₂—, R² is H—, and R³ is H—.

7. The method of claim 5, wherein R¹ is CH₃—, R² is PhCH₂—, and R³ is CH₃—.

8. The method of claim 5, wherein R¹ is CH₃—, R² is 6-CH₃-3-Py-(CH₂)₂— and R³ is H—.

9. The method of claim 5, where R¹ is CH₃—, R² is 6-CH₃-3-Py-(CH₂)₂— and R³ is CH₃—.

10. The method of claim 5, wherein R¹ is CH₃—, R² is H—, and R³ is H— or CH₃—.

11. The method of claim 5, wherein R¹ is CH₃—, R² is H—, and R³ is Br—.

12. The method of claim 5, wherein the pharmaceutically acceptable salt is a pharmaceutically acceptable acid salt.

13. The method of claim 5, wherein said compound is 2,8-dimethyl-5-[2-(6-methyl-pyridyl-3)-ethyl]-2,3,4,5-tetrahydro-1H-pyrido[4,3-b]indole dihydrochloride.

14-15. (canceled)

16. A kit comprising:
(1) a hydrogenated pyrido(4,3-b)indole of the formula (2), or a pharmaceutically acceptable salt thereof, 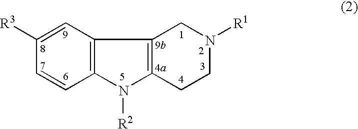
wherein:
R¹ is CH₃—, CH₃CH₂— or PhCH₃;
R² is H—, PhCH₂— or 6-CH₃-3-Py-(CH₂)₂—, and
R³ is H—, CH₃— or Br—; and
(2) instructions for use in the treatment of mild cognitive impairment.

17. The kit of claim 16, wherein the hydrogenated pyrido(4,3-b)indole is 2,8-dimethyl-5-(2-(6-methyl-3-pyridyl)ethyl)-2,3,4,5-tetrahydro-1H-pyrido[4,3-b]indole dihydrochloride.
